(19) United States
(12) Patent Application Publication
(10) Pub. No.: US 2015/0297120 A1
(43) Pub. Date: Oct. 22, 2015

(54) Method For Tracking Motion of Subject in Real Time and for Correcting Medical Image

(71) Applicant: GACHON UNIVERSITY OF INDUSTRY-ACADEMIC COOPERATION FOUNDATION, Gyeonggi-do (KR)

(72) Inventors: Young Don SON, Gyeonggi-do (KR); Hang Keun KIM, Incheon (KR); Zang Hee CHO, Incheon (KR); Young Bo KIM, GYEONGGI-DO (KR); Dae Hyuk KWON, SEOUL (KR); Yo Han JOO, Incheon (KR)

(73) Assignee: GACHON UNIVERSITY OF INDUSTRY-ACADEMIC COOPERATION FOUNDATION, Gyeonggi-do (KR)

(21) Appl. No.: 14/361,427

(22) PCT Filed: Apr. 17, 2013
(86) PCT No.: PCT/KR2013/003220

§ 371 Date: May 29, 2014

(30) Foreign Application Priority Data
Apr. 27, 2012 (KR) 10-2012-0044407

(51) Int. Cl. A61B 5/11; G06T 7/20; A61B 6/00; A61B 6/03; A61B 8/13; A61B 8/00; G06T 11/00; A61B 5/055

(52) U.S. Cl. CPC A61B 5/11; G06T 11/005; G06T 7/20; A61B 5/055; A61B 6/037; A61B 8/13; A61B 6/032; A61B 8/54; A61B 6/54

(57) ABSTRACT

The present invention relates to a method for tracking the motion of a subject in real time and for correcting a medical image. The method includes the steps of: (a) a motion detection module outputting real-time motion information of a subject; (b) a motion calculation module receiving and analyzing the real-time motion information, transforming the analyzed result into triaxial motion parameters, and outputting the triaxial motion parameters; (c) a driving module driving a driving motor in response to the triaxial motion parameters; and (d) a medical image data acquisition module moving by as much as the subject moves in response to the driving of the driving motor. According to the present invention, medical image data can be acquired as if a subject has not moved during imaging by calculating the triaxial motion parameters of a subject through motion information acquired from the motion detection module and moving the medical image data acquisition module according to the triaxial motion parameters. Therefore, the acquisition of an erroneous sample due to the movement of the subject can be minimized, and the precision of the medical image can be improved by simultaneously moving the medical image data acquisition module in real time according to the movement of the subject.

### TECHNICAL FIELD

The present invention relates to a method for correcting a medical image, and more particularly, to a method for tracking the motion of a subject in real time and for correcting a medical image, which is capable of directly moving a measurement instrument according to the motion of a subject after image data is acquired by a medical imaging device which needs to properly correct the motion of the subject in order to acquire a precise medical image, and correcting an error in real time while the image is acquired.

### BACKGROUND ART

The development of modern medical imaging devices has led to a lot of innovation in the medical profession. The medical imaging devices can analyze a brain structure and functional processes without damaging the human body, thereby contributing to the effective treatment of a lot of diseases. In particular, positron emission tomography (PET) can quantitatively measure energy metabolism, distribution of acceptors, the secreted amount of neurotransmitters, the degree of gene expression and the like according to a radioactive ligand used in the PET. Thus, the PET system has advantages which cannot be obtained by other medical imaging devices.

However, since the spatial resolution of the image is limited by the physical and structural characteristics of the detector used in the PET system, the resolution may be degraded in comparison to other imaging systems.

Furthermore, since the PET system detects decay of positrons emitted from the radioactive ligand, the PET system requires a long scan time of 30 minutes to obtain a sufficient number of samples.

During such a long scan time, the motion of a subject must be restricted as much as possible. However, when the subject unconsciously moves due to a physiological phenomenon, sampling occurs at the changed position. In this case, since data of the wrong position is used to restore an image, the image data may be inaccurately restored.

In general, as the scan time of a medical imaging device such as PET increases, a subject inevitably moves more. The influence of the motion further increases when the medical imaging device has a high resolution.

In medical imaging methods such as PET which need to perform quantitative measurement, it is especially important to reduce measurement error for an images caused by the motion. Thus, a process of properly correcting of the motion of the subject is required to acquire an accurate medical image.

Conventionally, motion information has been obtained through a system for detecting the motion of a subject. Then, the medical image is corrected based on the motion information by using an image reconstruction algorithm.

However, when the motion is corrected through software after the image data is acquired, error correction in a quantitative correction process for an image, such as attenuation, scattering, or normalization, as well as the software motion correction serve as important factors.

In order to overcome such problems, the obtained motion information needs to be utilized in hardware devices, and the motion may be corrected by moving the measurement instrument according to the motion of the subject.

### DISCLOSURE

### Technical Problem

An object of the present invention is to provide a method for tracking the motion of a subject in real time and for correcting a medical image, which detects and analyzes real-time motion information of a subject in a medical imaging device, acquires image data, and then directly moves a measuring instrument in real time according to the motion of the subject, thereby correcting the image quantitatively.

### Technical Solution

According to one aspect of the present invention, there is provided a method for tracking the motion of a subject in real time and for correcting a medical image. The method may include the steps of: (a) acquiring data containing real-time motion information of a subject, by a motion detection module; (b) receiving and analyzing the acquired data by a motion detection module, transforming the analyzed result into triaxial motion parameters, and outputting the triaxial motion parameters, by a motion calculation module; (c) driving motors of a medical image acquisition module in response to the triaxial motion parameters, by a driving module; and (d) moving the medical image data acquisition module by as much as the subject actually moves in response to the driving of the motors.

The method may include the step of transforming the triaxial motion parameters into machine codes, by a control module, after the step (b) and before the step (c).

The method may include the steps of, before the step (a): (e) acquiring medical image data of the subject, by the medical image data acquisition module; and (f) receiving the acquired data, reconstructing a medical image from the acquired data, and storing the reconstructed medical image, by a data processing module.

In the step (d), the driving module may drive the motors at both sides of the medical image data acquisition module in different directions so as to rotate the medical image data acquisition module by an estimated angle as much as the subject actually moves.

In the step (d), the driving module may drive the motors at both sides of the medical image data acquisition module in the same direction so as to horizontally move the medical image data acquisition module by an estimated distance as much as the subject actually moves.

The medical image data acquisition module may include any one of PET, MRI, SPECT, X-ray, CT, and Ultrasound devices.

The motion detection module may include any one of a CCD camera, a gyroscope, and a ray sensor.

### Advantageous Effects

In accordance with the present invention, the triaxial motion parameters of a subject may be calculated through motion information acquired from the motion detection module, and the medical image data acquisition module may be moved according to the triaxial motion parameters. Thus, medical image data can be acquired as if a subject has not moved. Therefore, the acquisition of an erroneous sample due to the movement of the subject can be minimized.

Furthermore, the precision of the medical image can be improved by simultaneously moving the medical image data acquisition module in real time according to the movement of the subject.

### DESCRIPTION OF DRAWINGS

FIG. 1 is a configuration diagram of an apparatus for tracking the motion of a subject in real time and for correcting a medical image in accordance with an embodiment of the present invention.

FIG. 2 is a flowchart illustrating a method for tracking the motion of a subject in real time and for correcting a medical image in accordance with an embodiment of the present invention.

FIGS. 3A and 3B are diagrams illustrating an example in which a medical image data acquisition module 110 is moved according to the method for tracking the motion of a subject in real time and for correcting a medical image in accordance with the embodiment of the present invention.

FIGS. 4A and 4B are diagrams illustrating an example in which the medical image data acquisition module 110 is moved by motors according to the method for tracking the motion of a subject in real time and for correcting a medical image in accordance with the embodiment of the present invention.

FIGS. 5A to 5C are diagrams illustrating an example in which the medical image data acquisition module 110 is moved on each plane of three-dimensional space by the motors according to the method for tracking the motion of a subject in real time and for correcting a medical image in accordance with the embodiment of the present invention.

### BEST MODE

Hereinafter, a method for tracking the motion of a subject in real time and for correcting a medical image in accordance with an embodiment of the present invention will be described with reference to the accompanying drawings.

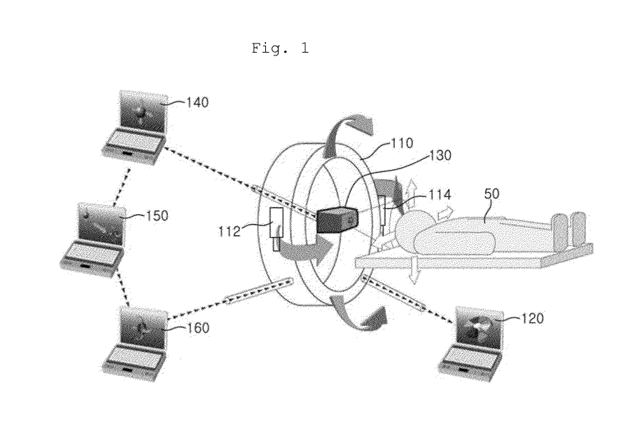

FIG. 1 is a configuration diagram of an apparatus for tracking the motion of a subject in real time and for correcting a medical image in accordance with an embodiment of the present invention. The apparatus includes a medical image data acquisition module 110, a data processing module 120, a motion detection module 130, a motion calculation module 140, a control module 150, and a driving module 160.

FIG. 2 is a flowchart illustrating a method for tracking the motion of a subject in real time and for correcting a medical image in accordance with an embodiment of the present invention.

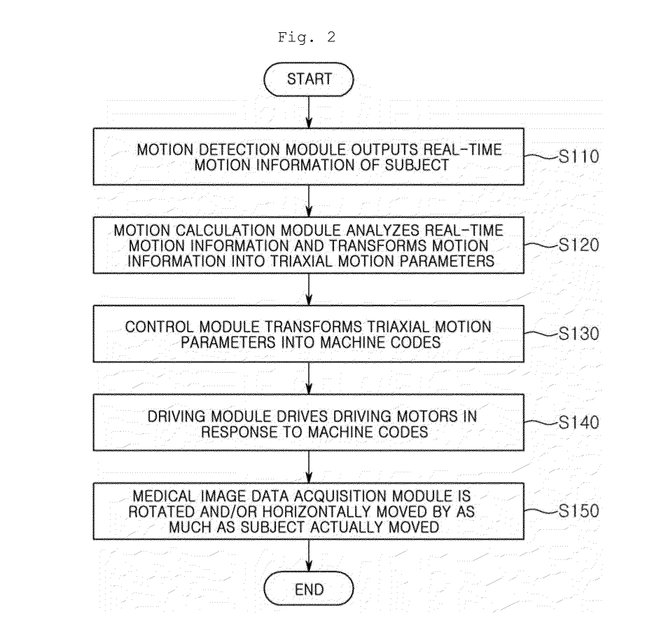

FIGS. 3A and 3B are diagrams illustrating an example in which the medical image data acquisition module 110 is moved according to the method for tracking the motion of a subject in real time and for correcting a medical image in accordance with the embodiment of the present invention. FIG. 3A illustrates the case in which the subject rotates by an estimated angle A_{sub}, and FIG. 3B illustrates the case in which the subject horizontally moves by an estimated distance D_{sub}.

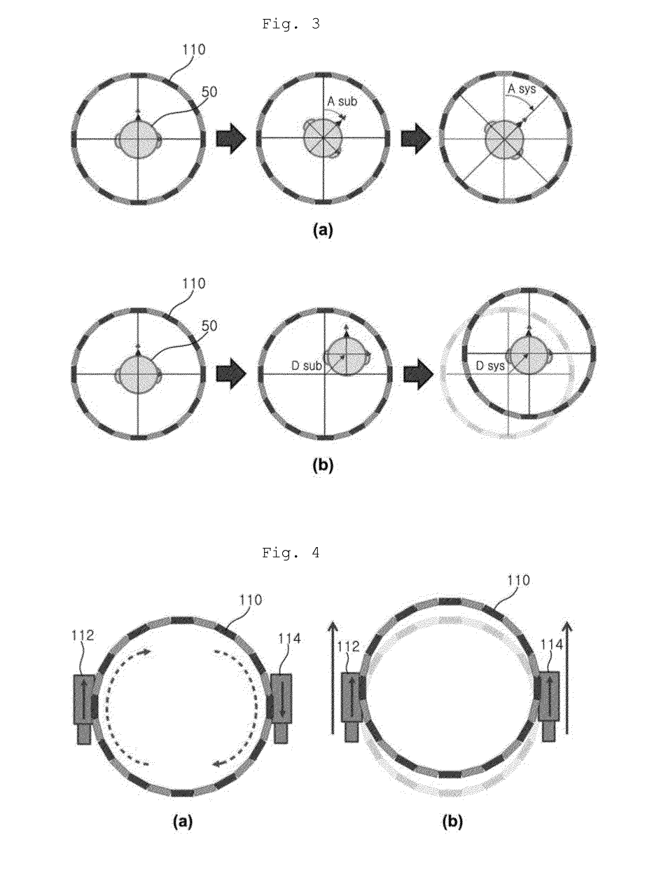

FIGS. 4A and 4B are diagrams illustrating an example in which the medical image data acquisition module 110 is moved by motors 112 and 114 according to the method for tracking the motion of a subject in real time and for correcting a medical image in accordance with the embodiment of the present invention. FIG. 4A illustrates the case in which the medical image data acquisition module 110 is rotated, and FIG. 4B illustrates the case in which the medical image data acquisition module 110 is horizontally moved.

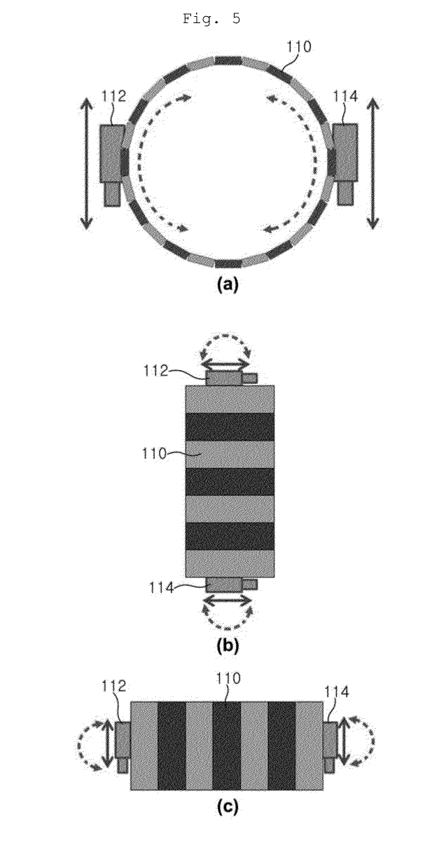

FIGS. 5A to 5C are diagrams illustrating an example in which the medical image data acquisition module 110 is moved on each plane of three-dimensional space by the motors 112 and 114 according to the method for tracking the motion of a subject in real time and for correcting a medical image in accordance with the embodiment of the present invention. FIG. 5A illustrates the motion on the X-Y plane, FIG. 5B illustrates the motion on the Y-Z plane, and FIG. 5C illustrates the motion on the Z-X plane.

Referring to FIGS. 1 to 5, the method for tracking the motion of a subject in real time and for correcting a medical image according to the embodiment of the present invention will be described.

As soon as a scanning operation for a medical image is started, the medical image data acquisition module 110 and the motion detection module 130 start operating.

The medical image data acquisition module 110 is a mechanical and electronic equipment including a sensor unit required for acquiring data of a medical image to be measured. The medical image data acquisition module 110 may include PET, MRI, SPECT, X-ray, CT, Ultrasound devices and the like.

The data processing module 120 receives the acquired data of the subject from the medical image data acquisition module 110, reconstructs an image, and stores the reconstructed image.

At this time, when the subject 50 moves, the motion detection module 130 detects the motion of the subject and outputs real-time motion information at step S110. The motion detection module 130 may include a CCD camera, a gyroscope, or a ray sensor depending on the sensor.

In general, the motion of a solid object may be expressed as six motion parameters including triaxial linear movements and triaxial rotational movements. The motion calculation module 140 receives the real-time motion information from the motion detection module 130, analyzes the received information, and transforms the analyzed information into six motion parameters DOF at step S120.

The control module 150 receives the motion parameters from the motion calculation module 140 and transforms the received motion parameters into machine codes at step S130. The driving module 160 drives motors 112 and 114 for the medical image data acquisition module 110 in response to the machine codes transformed by the control module 150 at step S140. Then, the medical image data acquisition module 110 is moved by as much as the subject moved, in six directions based on a combination of linear motions and rotational motions at step S150.

At this time, in order for the driving module 160 to drive the motors 112 and 114 to move the medical image data acquisition module 110, the medical image data acquisition module 110 needs to have a structure which can be rotated and moved along three axes.

For this structure, the apparatus for tracking the motion of a subject in real time and for correcting a medical image according to the embodiment of the present invention includes the motors 112 and 114 installed on the medical image data acquisition module 110 corresponding to the axis. The motors 112 and 114 may move in both directions.

That is, as illustrated in FIGS. 4A and 4B, when the motors 112 and 114 move in different directions from each other, the medical image data acquisition module 110 may be rotated, and when the motors 112 and 114 are operated in the same direction, the medical image data acquisition module 110 may be axially moved.

Furthermore, as illustrated in FIGS. 5A to 5B, the medical image data acquisition module 110 may be horizontally moved and rotated in the triaxial directions according to the operation directions of the motors 112 and 114 for the medial image data acquiring module 110 on the three-dimensional planes (X-Y, Y-Z, and Z-X).

For example, as illustrated in FIG. 3A, suppose that the subject 50 is rotated by an estimated angle A_{sub} while the medical image data acquisition module 110 is scanning a medical image of the subject 50.

When the motion detection module 130 detects the motion of the subject 50 and outputs real-time motion information, the motion calculation module 140 receives the real-time motion information, indicating that the subject 50 rotated at the estimated angle A_{sub}, from the motion detection module 130, analyzes the received information, and transforms the analyzed information into three motion parameters for rotational movement and three motion parameters for horizontal movement.

The control module 150 receives the six motion parameters from the motion calculation module 140, and transforms the received motion parameters into machine codes, and the driving module 160 drives the motors 112 and 114 for the medical image data acquisition module 110 in response to the machine codes obtained by transforming the motion parameters through the control module 150, moves the motors 112 and 114 in different directions as illustrated in FIG. 4A, and rotates the medical image data acquisition module 110 at the same angle A_{sys} as the estimated angle A_{sub} the subject 50 rotated, as illustrated in FIG. 3A.

Furthermore, as illustrated in FIG. 3B, suppose that the subject 50 horizontally moves by an estimated distance D_{sub} while the medical image data acquisition module 110 is scanning a medical image of the subject 50.

When the motion detection module 130 detects the motion of the subject 50 and outputs real-time motion information, the motion calculation module 140 receives the real-time motion information, indicating that the subject 50 horizontally moved by the estimated distance D_{sub}, from the motion detection module 130, analyzes the received information, and transforms the analyzed information into three motion parameters for rotational movement and three motion parameters for horizontal movement.

The control module 150 receives the six motion parameters from the motion calculation module 140 and transforms the received motion parameters into machine codes, and the driving module 160 drives the motors 112 and 114 at both sides of the medical image data acquisition module 110 in response to the machine codes obtained by transforming the six motion parameters through the control module 150, moves the motors 112 and 114 in the same direction, as illustrated in FIG. 4B, and horizontally moves the medical image data acquisition module 110 by the same distance D_{sys} as the estimated distance D_{sub} the subject 50 actually moved, as illustrated in FIG. 3B.

The series of operations are performed in real time until the imaging operation for the subject 50 is completed.

At this time, two or more motors may be installed at each axis so as to drive the module. In this case, a driving unit (not illustrated) including the motors needs to be designed to endure the weight of the medical image data acquisition module 110 and precisely move in the order of milimiters.

As such, the method for tracking the motion of a subject in real time and for correcting a medical image according to the embodiment of the present invention may calculate the triaxial motion parameters of the subject 50 through the motion information obtained from the motion detection module 130, move the medical image data acquisition module 110 in real time, and acquire medical image data as if the subject did not move during the imaging operation. Thus, the method may minimize erroneous samples caused by the movement of the subject 50.

Furthermore, since the medical image data acquisition module 110 is simultaneously moved in real time according to the movement of the subject 50, the precision of the acquired medical image is improved.

Each element of the present invention can be implemented as a single independent unit or combined together to form an integrated unit, and some elements can be omitted according to some configurations. Various modifications and changes can be made without departing from the scope of the invention.

1. A method for tracking the motion of a subject in real time and for correcting a medical image, comprising the steps of:
(a) acquiring data containing real-time motion information of a subject, by a motion detection module;
(b) receiving and analyzing the acquired data by a motion detection module, transforming the analyzed result into triaxial motion parameters, and outputting the triaxial motion parameters, by a motion calculation module;
(c) driving motors of a medical image acquisition module in response to the triaxial motion parameters, by a driving module; and
(d) moving the medical image data acquisition module by as much as the subject actually moves in response to the driving of the motors.

2. The method of claim 1, further comprising the step of transforming the triaxial motion parameters into machine codes, by a control module, after the step (b) and before the step (c).

3. The method of claim 2, further comprising the steps of, before the step (a):
(e) acquiring medical image data of the subject, by the medical image data acquisition module; and
(f) receiving the acquired data, reconstructing a medical image from the acquired data, and storing the reconstructed medical image, by a data processing module.

4. The method of claim 1, wherein, in the step (d),
the driving module drives the motors at both sides of the medical image data acquisition module in different directions so as to rotate the medical image data acquisition module by an estimated angle as much as the subject actually moves.

5. The method of claim 1, wherein, in the step (d),
the driving module drives the motors at both sides of the medical image data acquisition module in the same direction so as to horizontally move the medical image data acquisition module by an estimated distance as much as the subject actually moves.

6. The method of claim 1, wherein the medical image data acquisition module comprises any one of PET, MRI, SPECT, X-ray, CT, and Ultrasound devices.

7. The method of claim 1, wherein the motion detection module comprises any one of a CCD camera, a gyroscope, and a ray sensor.
